(19) United States
(12) Patent Application Publication
(10) Pub. No.: US 2021/0407679 A1
(43) Pub. Date: Dec. 30, 2021

(54) DEEP-LEARNING BASED CERTAINTY QUALIFICATION IN DIAGNOSTIC REPORTS

(71) Applicant: University of Massachusetts, Boston, MA (US)

(72) Inventors: Feifan Liu, Worcester, MA (US); Max P. Rosen, Weston, MA (US)

(73) Assignee: University of Massachusetts, Boston, MA (US)

(21) Appl. No.: 17/358,481

(22) Filed: Jun. 25, 2021

Related U.S. Application Data
(60) Provisional application No. 63/043,946, filed on Jun. 25, 2020.

(51) Int. Cl. G16H 50/20; G06N 3/08; G16H 15/00; G06K 9/62; G06F 40/20

(52) U.S. Cl. CPC G16H 50/20; G06N 3/088; G06F 40/20; G06K 9/6265; G16H 15/00

(57) ABSTRACT

A method for assessing diagnostic certainty in diagnostic reporting natural language, the method comprising receiving a natural language impression portion of a diagnostic report submitted for certainty evaluation, the impression portion having one or more sentences of natural language, accessing a pre-trained and fine-tuned language model, applying the one or more sentences to the trained language model for evaluation of the one or more sentences as a whole, receiving an assessment of certainty for the respective one or more sentences, based on the evaluation, communicating the assessment of certainty to a user before accepting the impression portion, and accepting submission of the impression portion only after the impression portion satisfies certainty criteria, or if the certainty criteria is not required obtaining validation from the user.

### BACKGROUND

### 1. Field of the Disclosure

The present disclosure relates to document processing of using machine learning, and more particularly, to deep-learning based certainty qualification in diagnostic reports.

### 2. Description of Related Art

Diagnostic ambiguity expressed in radiology reports can undermine the effectiveness of the radiology report and can contribute to overutilization of follow-up imaging studies, delayed patient care, or inappropriate treatment.

Governing bodies, such as the American College of Radiology (ACR), have acknowledged that there is a need for precision in communication within diagnostic reports, for example in radiology reports. Clarity by referring physicians can be a useful quality metric of radiological reports. However, a referring physician can interpret the level of confidence associated with free text expressions used by a radiologist to be different from a level of diagnostic confidence the radiologist intended to convey. Examples of reporting characteristics that can undermine effectiveness of a radiology report include overuse of hedging terms and provision of a longer-than-expected list of multiple differential diagnoses. A radiology that lacks certainty can contribute to overutilization of follow-up imaging studies, delayed patient care, and/or inappropriate treatment.

One proposed solution uses a standardized lexicon. However, using a restricted lexicon can be limited to assisting with lexical-level interpretation of one word at a time, which loses diagnostic confidence in the radiology report that is context-dependent. For example, the same term used for diagnostic certainty can have different interpretations based on how differential diagnoses were reported in the context. For example, “likely to be Arnold Chiari 1 malformation” indicates mild certainty of a diagnosis, while “likely differential considerations include demyelinating/inflammatory processes” indicates uncertainty of the diagnosis. Additionally, application of a standardized lexicon is not designed to mitigate overuse of hedge words when reporting a diagnosis that could be reported with greater certainty.

Natural language processing (NLP) is an artificial intelligence (AI) technology that uses linguistic and statistical approaches to understand the semantics of free texts. NLP has been widely applied to radiology research for automatic identification and extraction of clinically significant information. However application of NLP and/or AI for uncertainty analysis is limited to the lexicon level (e.g., to detect hedging cue terms), is overly dependent on hedging terms, and/or provides binary detection of uncertainty that do not provide both qualitative and quantitative assessments of uncertainty.

While conventional methods and systems have generally been considered satisfactory for their intended purpose, there is still a need in the art for certainty assessments of diagnostic reports systems and methods that can quantify and qualify certainty based on context.

### SUMMARY

The purpose and advantages of the below described illustrated embodiments will be set forth in and apparent from the description that follows. Additional advantages of the illustrated embodiments will be realized and attained by the devices, systems and methods particularly pointed out in the written description and claims hereof, as well as from the appended drawings.

To achieve these and other advantages and in accordance with the purpose of the illustrated embodiments, in one aspect, disclosed is a method for assessing diagnostic certainty in diagnostic reporting natural language. The method includes receiving an impression portion of a diagnostic report submitted for certainty evaluation, wherein the impression portion has one or more sentences of natural language. The method further includes accessing a trained language model, wherein the trained language model was trained in a pre-training stage and in a fine-tuning stage.

In the pre-training stage, the trained language model was trained in an unsupervised manner using artificial intelligence-based deep neural network learning by deep bidirectional reading of a large amount of word sequences of first training sentences of natural language and outputting a bidirectional language model, wherein the language model is configured to predict one or more words from their respective bidirectional context and to transform natural language input into a latent semantic space to represent the respective one or more words based on bi-directionally surrounding words.

In the fine-tuning stage, the trained language model was trained for evaluating certainty of a small amount of training impression portions of diagnostic reports specific to a task, wherein the training impression portions include a plurality of one or more second training sentences of natural language. Evaluating the certainty of the training impression portions includes generating certainty data per second training sentence indicative of a result of applying annotation rules specific to the task based on context provided by the second training sentence as a whole.

The method further includes applying the one or more sentences to the trained language model for evaluation of the one or more sentences as a whole, receiving an assessment of certainty for the respective one or more sentences, communicating the assessment of certainty to a user before accepting the impression portion, and accepting submission of the impression portion only after the impression portion satisfies certainty criteria, or if the certainty criteria is not required, obtaining validation from the user.

In one or more embodiments, the method can further include generating the assessment of certainty, wherein the assessment of certainty includes assignment to a certainty category of a plurality of certainty category, each certainty category indicating a different level or type of certainty.

In one or more embodiments, generating the assessment of certainty can further include determining a probability that the assignment to the certainty category is correct.

In one or more embodiments, the method can further include, for an assessment of certainty that fails to satisfy a certainty criteria, providing an opportunity to update the impression portion and resubmitting the impression portion for application of the one or more sentences of the updated impression portion to the trained language model.

In one or more embodiments, the method can further include training the trained language model in the fine-tuning stage using a training set that is a subset of the certainty data.

In one or more embodiments, training the trained language model in the fine-tuning stage can include at least one of validating using a validation set that is a subset of the certainty data and testing using a testing set that is a subset of the certainty data.

In one or more embodiments, training the trained language model in the fine-tuning stage can include iteratively adjusting at least one of the annotation rules and the certainty data by a plurality of reviewers until evaluation of same second training sentences by the plurality of reviewers results in the certainty data generated by different reviewers of the plurality of reviewers satisfying a criterion of consensus.

In one or more embodiments, the method can further include retraining the trained language model in the fine-tuning stage, based on a state of at least a portion of the trained language model after the pre-training stage and before the fine-tuning stage, using a second small amount of second training impression portions of diagnostic reports specific to a second task, the second training impression portions including a plurality of one or more third training sentences of natural language, the evaluating certainty of the second training impression portions including generating second certainty data per third training sentence indicative of a result of applying annotation rules specific to the second task based on context provided by the third training sentence as a whole.

In accordance with further aspects of the disclosure, one or more computer systems are provided that include a memory configured to store instructions and a processor disposed in communication with the memory, wherein the processor upon execution of the instructions is configured to perform each of the respective disclosed methods. In accordance with still further aspects of the disclosure non-transitory one or more computer readable storage mediums and one or more computer programs embedded therein are provided, which when executed by a computer system, cause the computer system(s) to perform the respective disclosed methods.

These and other features of the systems and methods of the subject disclosure will become more readily apparent to those skilled in the art from the following detailed description of the preferred embodiments taken in conjunction with the drawings.

### BRIEF DESCRIPTION OF THE DRAWINGS

So that those skilled in the art to which the subject disclosure appertains will readily understand how to make and use the devices and methods of the subject disclosure without undue experimentation, preferred embodiments thereof will be described in detail herein below with reference to certain figures, wherein:

FIG. 1 shows a schematic view of an exemplary embodiment of a diagnostic report processing system in accordance with embodiments of the disclosure;

FIG. 2 shows a block diagram of an exemplary system for generating annotated data used by the diagnostic report processing system in accordance with embodiments of the disclosure;

FIG. 3 shows a schematic diagram of pin accordance with embodiments of the disclosure;

FIG. 4 shows a flowchart of an example method of assessing certainty of diagnostic reports, in accordance with embodiments of the disclosure;

FIG. 5 shows an example method of generating annotation data, in accordance with embodiments of the disclosure;

FIG. 6 shows an example method of training a model used by the diagnostic report processing system, in accordance with embodiments of the disclosure;

FIG. 7 shows a flow diagram 700 of an example method of processing an example sentence while fine-tuning a pre-trained bidirectional encoder representations from transformers (BERT) model, in accordance with embodiments of the disclosure;

FIG. 8A shows performance curves over a number of epochs using validation annotation data during a fine-tuning process of a pre-trained BERT-base model;

FIG. 8B shows performance curves over a number of epochs using validation annotation data during a fine-tuning process of a pre-trained bioBERT model;

FIG. 8C shows area under the receiver operating characteristic curve (ROC-AUC) using testing annotation data during a fine-tuning process of different pre-trained BERT models; and

FIG. 9 shows a block diagram of an exemplary computer system configured to implement components of the diagnostic report processing system, in accordance with embodiments of the disclosure.

### DETAILED DESCRIPTION OF THE PREFERRED EMBODIMENTS

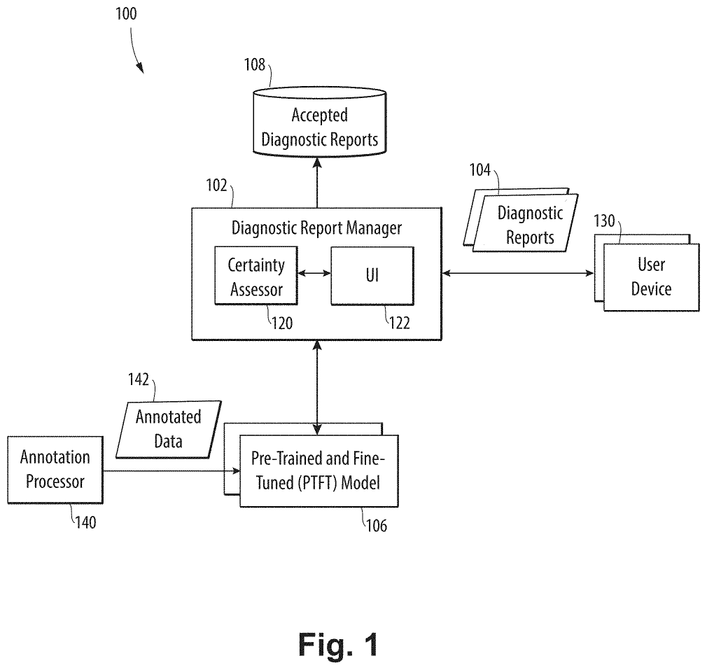

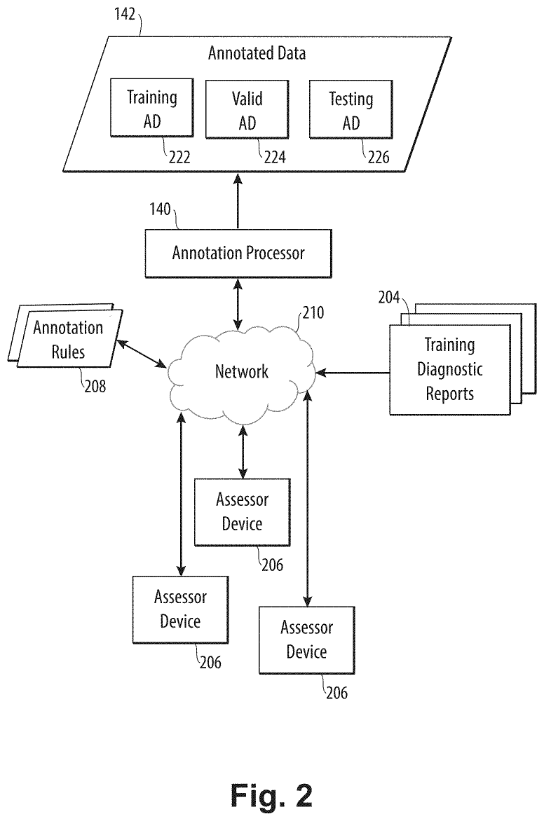

Reference will now be made to the drawings wherein like reference numerals identify similar structural features or aspects of the subject disclosure. For purposes of explanation and illustration, and not limitation, a block diagram of an exemplary embodiment of a diagnostic report processing system 100 accordance with the disclosure is shown in FIG. 1 and is designated generally by reference character 100. Methods associated with operations of the diagnostic report processing system 100 in accordance with the disclosure, or aspects thereof, are provided in FIGS. 2-9, as will be described. The systems and methods described herein can be used to apply deep learning when processing diagnostic reports to assess the diagnostic reports for certainty based on context for both quantification and qualification of certainty.

Diagnostic ambiguity expressed in diagnostic reports can undermine effectiveness of a diagnostic report and can contribute to overutilization of follow-up diagnostic studies, delayed patient care, or inappropriate treatment. Diagnostic report processing system 100 addresses this challenge by applying artificial intelligence (AI), using deep transfer learning in an automated fashion to unlock contextualized semantics and determine a certainty classification using limited annotated data. Certainty classification can be performed retrospectively and/or prospectively. Interactive features of diagnostic report processing system 100 can interact with practitioners to by providing feedback to prompt practitioners to improve precision of diagnostic findings in their reports and/or reject diagnostic reports that do not meet threshold standards of certainty.

Diagnostic report processing system 100 uses a pre-trained bidirectional encoder representations from transformers (BERT) model that is fine-tuned using task-specific annotated data to provide a deep transferring learning model. This model can be applied to diagnostic reports to determine contextualized certainty at the sentence level. Task-specific refers to a task of determining certainty in diagnostic reporting associated with a specific field, such as a particular part of the anatomy (e.g., brain, pulmonary organs, etc.) and/or using a specific diagnostic modality (e.g., magnetic resonance imaging (MRI), computer tomography (CT), ultrasound, etc.).

Diagnostic report processing system 100 includes a diagnostic report manager 102 that receives diagnostic reports 104 from a user device 130. Diagnostic report manager 102 includes a certainty assessor 120 and a user interface (UI) 122. Certainty assessor 120 assesses received certainty diagnostic reports 104. The diagnostic reports 104 can be submitted via user device 130 or received via a different source (such as a processing device external or internal to diagnostic report processing system 100). Certainty assessor 120 uses a pre-trained and fine-tuned (PTFT) model 106.

PTFT model 106 is first formed from a pre-trained BERT model. For example, the pre-trained BERT model can be imported into PTFT model 106. During a pre-training process, the BERT model learns deeper and intimate understandings of how a language works. The pre-training process uses large volumes of plain text data in an unsupervised manner. Since the BERT model reads a sequence of words (e.g., a sentence) bi-directionally, the sequence of words is read as a single unit, as opposed to multiple units composed of terms or phrases. This characteristic allows the Bert model during pre-training to learn the context of a word in the sequence of words based on its surroundings. The pre-trained Bert model can thus receive natural language input and predict any word from its bidirectional language model. The pre-trained Bert model can further encode the sequence of words into a latent semantic space, such as a numeric vector that represents the respective words based on words surrounding the word in either direction (also referred to as bidirectional surrounding words).

During a fine-tuning process, parameters of the pre-trained BERT model are fine-tuned (before or after importation) using annotated data 142 to build PTFT model 106 into a task-specific model. An annotation processor 140 (described in greater detail with respect to FIG. 2) is configured to provide the annotated data 142. Word sequences having multiple terms are used as input for the fine-tuning process. In the example shown, the word sequences are sentences. Tokens are applied to words, sentences boundaries are identified, and certainty labels are appended to the sentences, if any. Words can be broken into sub-words (e.g., prefix, root, suffix) to mitigate out-of-vocabulary issues. In one or more embodiments, sentences characterized as being short (e.g., having below a threshold number of words) are removed for potentially introducing noise. In one or more embodiments, a classification label [CLS] is added at the beginning of each sentence, and a separation label [SEP] is added at the end of each sentence to separate multiple sentences.

The fine-tuning process includes initializing n transformer encoders of the pre-trained BERT model. After initialization, parameters of the pre-trained Bert model, including parameters of its fully connected layer, are fine-tuned for certainty classification through supervised learning using the annotated data 142. By applying an encoder of the pre-trained BERT, wherein the encoder includes a multi-layer deep neural network architecture, each classification token that is included with the input is transformed to a vector representation, wherein the vector representation represents a final hidden state.

A sentence classification task further includes feeding a final hidden state of the classification token appended to a sentence into a fully-connected layer, wherein the classification token represents the complete sentence to which it is appended. The fully-connected layer processes the classification token to determine a probability distribution over several possible certainty categories.

UI 122 can receive data to be analyzed from a user device 130. The data can include a diagnostic report or one or more portions of the diagnostic report, such as an impression portion. The impression portion is a textual portion of the diagnostic report in which a diagnostician's findings are summarized. Of particular interest in the impression portion is a diagnosis explanation that explains a condition that may be causing a problem. The diagnosis explanation can then be used for making decisions, such as which treatment plan to use or whether further testing is needed. Data received from UI 122 is provided to certainty assessor 120, which can filter the diagnosis explanation from the data, if needed. Sentences of the diagnosis explanation are provided as input to PTFT model 106.

Certainty assessor 120 can receive from PTFT model 106 a certainty classification for the diagnosis explanation per sentence or for the entire diagnosis explanation. A hierarchical structured model can be trained to integrate sentence-level information for multiple sentences of the diagnosis explanation to determine a certainty classification for the overall diagnosis explanation. The certainty classification provided can include a probability distribution over several possible certainty categories per sentence or for the entire diagnosis explanation.

Certainty assessor 120 can determine whether the certainty classification assigned to the diagnosis explanation and/or individual sentences of the diagnosis explanation satisfy a certainty criterion. Certainty assessor 120 decides, based on this determination, how the diagnosis report and/or associated diagnosis reports shall be treated. For example, depending on whether the certainty criterion is satisfied, certainty assessor 120 can decide whether to store the diagnosis explanation with the associated diagnostic report in a database of accepted diagnostic reports 108 and/or allow access to the diagnostic explanation for purposes of following through with recommendations in the diagnosis explanation. The database of accepted diagnostic reports 108 can be stored in a storage medium which can be included in, or separate from, diagnostic report manager 102. Following through can include allowing access by or submitting the diagnosis explanation to other parties or systems. Such other parties or systems can include a medical practitioner, a medical records system, a scheduler system for scheduling a follow-up appointment or a recommended follow-up procedure, an insurance claim processing system, etc.

When the certainty criterion is not satisfied, certainty assessor 120 can send a prompt to user device 130 to prompt a user to update the diagnostic explanation in order to satisfy the certainty criterion. The prompt can identify or imply a reason that the certainty criterion was not satisfied.

In one or more embodiments, when determining treatment of associated diagnosis reports, a proficiency grade can be associated with the author of the diagnostic explanation based on whether one or more criteria are satisfied by the certainty classification and/or the probability distribution for the diagnostic explanation. The proficiency grade can provide a quality metric that evaluates performance of the author with respect to expressing certainty, and can affect treatment of current, future, and/or past diagnostic explanations by the author. Treatment affected can include storage in the database of accepted diagnostic reports 108 and/or allowing access by or submitting the diagnosis explanation to other parties or systems. Furthermore, the proficiency grade can affect whether the one or more criteria are adjusted and/or whether the author is prompted to pass additional automated checkpoints in order for the author's future diagnostic explanations to be stored and/or accessible by other parties or systems. An example of an additional automated checkpoint is a requirement that the author use a checklist or questionnaire before submitting a future diagnosis explanation.

With reference to FIG. 2, a diagram of an example annotation processor 140 that generates annotated data 142 is shown. Each assessor user of multiple assessor users (without any limitation to a specific number of assessor users) operates a respective assessor device 206. The assessor user and/or the assessor device 206 can filter, if necessary, a portion of the diagnostic reports 204 that is being assessed, such as the diagnostic explanation. The assessor user accesses and evaluates training diagnostic reports 204 using annotation rules 208. The annotation rules 208 take into account the context of each sentence in diagnosis explanations of the diagnostic reports 204. The assessor user generates user-annotated data for each diagnosis explanation and submits the user annotated data via the assessor device 206 being used to annotation processor 140. The multiple assessor users use the same set of annotation rules 208 and evaluate the same diagnostic reports 204.

Using a first set of the training diagnostic reports 204 (which is a relatively small set), the assessor users perform an iterative process of adjusting the annotation rules 208 and amending the user-annotated data until a consensus is reached by the multiple assessor users. A consensus refers to the user-annotated data submitted by the different assessor users of the multiple assessor users for the respective training diagnostic reports 204 being sufficiently similar based on similarity criteria.

The assessor users then evaluate a second set of the training diagnostic reports 204 (which is larger than the first set) using the adjusted annotation rules 208. The process continues until a consensus is reached. Achievement of a consensus can be demonstrated, for example, by the user annotated data associated with the respective second set of training diagnostic reports 204 satisfying a similarity criteria (which can be the same or different than the similarity criteria used for the first set). If the similarity criteria is satisfied, then the user-annotated data can be submitted for use by the annotation processor 140, and the annotation guideline rules are fixed. The assessor users can use the annotation guideline rules to generate additional user annotated data by evaluating a third set of the training diagnostic reports 204 (which is much larger than the second set).

The assessor devices 206 can access the training diagnostic reports 204 and the annotation rules 208 and submit the user annotated data to annotation processor 140 via a network 210. Network 210 can be a local area network (LAN), a general wide area network (WAN), and/or a public network (e.g., the Internet). Communication via the network 210 with other computing devices can use wired or wireless communication links.

Annotation processor 140 can determine whether a consensus is met during evaluation of the first and second sets of the training diagnostic reports 204 and instruct the assessor users and/or their assessor devices 206 whether to repeat the iterative process. Annotation processor 140 further gathers the user-annotated data and outputs annotated data to PTFT model 106 (as shown in FIG. 1). The annotated data provided to PTFT model 106 can be divided into training annotated data (AD) 222, validation (valid) AD 224, and testing AD 226.

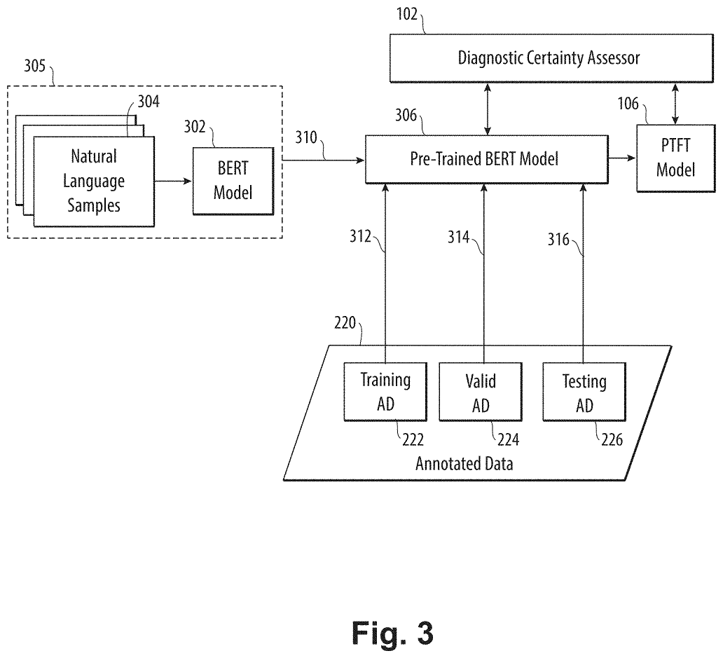

With reference to FIG. 3, a flow diagram is shown that demonstrates an example method of fine-tuning a pre-trained BERT model 306, which prepares the pre-trained BERT model 306 to become the PTFT model 106. The importation of the pre-trained BERT model 306 and fine-tuning method can be overseen by diagnostic report manager 102 or by a different processing device (not shown). Box 305 is dotted to show that a BERT model 302 is pre-trained using a very large set of natural language samples 304 (e.g., more than 10 billion words), resulting in pre-trained BERT model 306. The pre-trained BERT model 306 can be a publicly available model that is imported at importation operation 310. Next, the pre-trained BERT model 306 is fine-tuned using annotated data 142. The fine-tuning method can be performed in stages, which in the example correspond to fine-tuning operations 312, 314, and 316, resulting in PTFT model 106. Fine-tuning operation 312 uses the training AD 222, which comprises the bulk of annotated data 142, to fine-tune pre-trained Bert model 304 into a task-specific model using deep transfer leaning. In an example, without limitation to particular distribution of the annotated data 142, the annotated data 142 is distributed as follows: training AD 222 (80%), validation AD 224 (10%), and testing AD 226 (10%). Fine-tuning operation 314 uses the validation AD 224 to validate the training performed at operation 222. Fine-tuning operation 316 uses the testing AD to test the training and validation performed at fine-tuning operations 222 and 224. Fine-tune operations 222, 224 and/or 226 can be repeated until fine-tuning operation 226 provides satisfactory results.

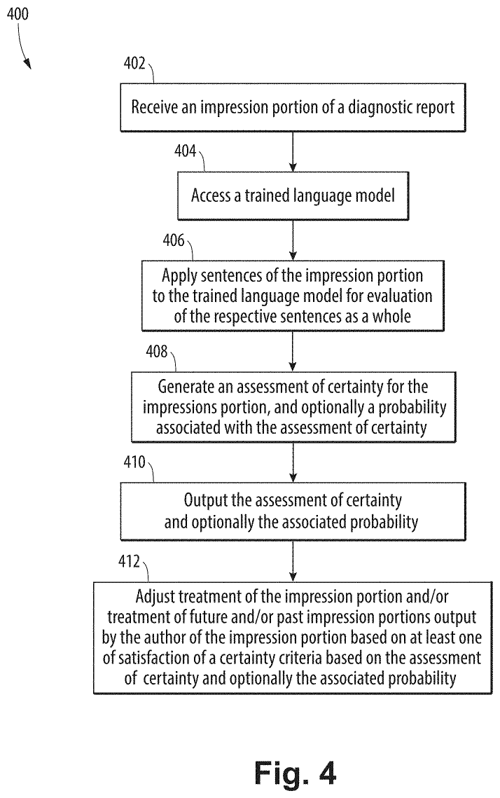

With reference now to FIGS. 4-6, shown are flowcharts demonstrating implementation of the various exemplary embodiments. It is noted that the order of operations shown in FIGS. 4-6 is not required, so in principle, the various operations may be performed out of the illustrated order. Also certain operations may be skipped, different operations may be added or substituted, some operations may be performed in parallel instead of strictly sequentially, or selected operations or groups of operations may be performed in a separate application following the embodiments described herein.

Language that refers to the exchange of information is not meant to be limiting. For example, the term “receive” as used herein refers to obtaining, getting, accessing, retrieving, reading, or getting a transmission. Use of any of these terms is not meant to exclude the other terms. Data that is exchanged between modules can be exchanged by a transmission between the modules, or can include one module storing the data in a location that can be accessed by the other module.

FIG. 4 shows a flowchart 400 of operations performed by a diagnostic report manager, such as diagnostic report manager 102 shown in FIG. 1. At operation 402, an impression portion of a diagnostic report is received (or accessed). The entire diagnostic report can be received and the impression portion can be accessed, or a particular portion of the impression portion (such as a diagnosis explanation) can be received or accessed. Operation 402 can include extracting impression portions (or portions of interest of impression portions) from the diagnostic reports (such as diagnostic reports 104 shown in FIG. 1) and converting the impression portions into plain text.

At operation 404, a trained language model is accessed. The trained language model can be a model such as PTFT model 106, shown in FIG. 1. The trained language model has been trained in a pre-training stage in an unsupervised manner using artificial intelligence-based deep neural network learning by deep bidirectional reading of a large amount of word sequences of first training sentences of natural language and outputting a measurement of certainty per first training sentence. The trained language model has been further trained in a fine-tuning stage for evaluating certainty of a small amount of training impression portions (or a portion of interest, such as a diagnostic explanation) of diagnostic reports specific to a task. The training impression portions include a plurality of one or more second training sentences of natural language. Evaluating certainty of the training impression portions includes generating certainty data per second training sentence indicative of a result of applying annotation rules specific to the task based on context provided by the second training sentence as a whole.

At operation 406, sentences of the impression portion are applied to the trained language model for evaluation of the respective sentences as a whole. At operation 408, an assessment of certainty is generated for the impressions portion, and optionally a probability associated with the assessment of certainty. At operation 410, the assessment of certainty is output, including optionally output of the associated probability. At operation 412, an adjustment is made to treatment of the impression portion and/or treatment of future and/or past impression portions output by the author of the impression portion based on at least one of satisfaction of a certainty criteria based on the assessment of certainty and optionally the associated probability.

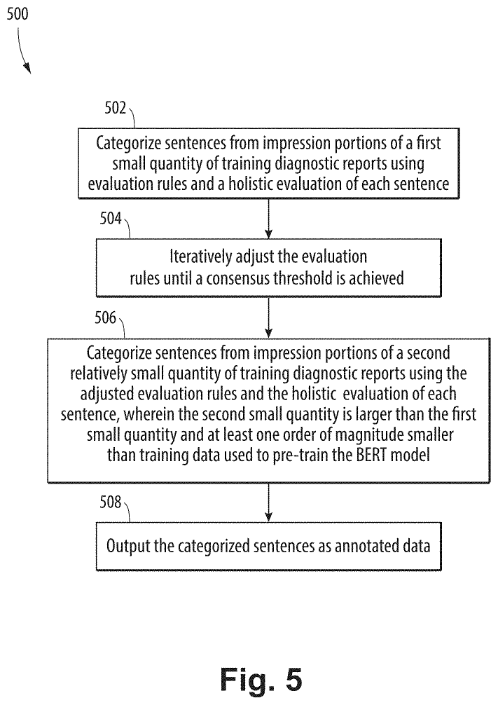

FIG. 5 shows a flowchart 500 of operations performed by an annotation processor, such as annotation processor 140 shown in FIG. 1. At operation 502, sentences from impression portions of a first small quantity of training diagnostic reports are categorized using annotation rules and a holistic evaluation of each sentence. The term “holistic” refers to considering the sentence as a whole, rather than as an assortment of unrelated of individual terms, in order to take into consideration the context provided by the sentence. This can include extracting impression portions (or portions of interest of impression portions) from training diagnostic reports (such as training diagnostic reports 204 shown in FIG. 2) and converting the impression portions into plain text. At operation 504, the annotation rules are iteratively adjusted until a consensus threshold is achieved. At operation 506, sentences from impression portions of a second relatively small quantity of training diagnostic reports are categorized using the adjusted annotation rules and the holistic evaluation of each sentence. The second small quantity is larger than the first small quantity and at least one order of magnitude smaller than training data used to pre-train the BERT model. At operation 508, the categorized sentences are output as annotated data.

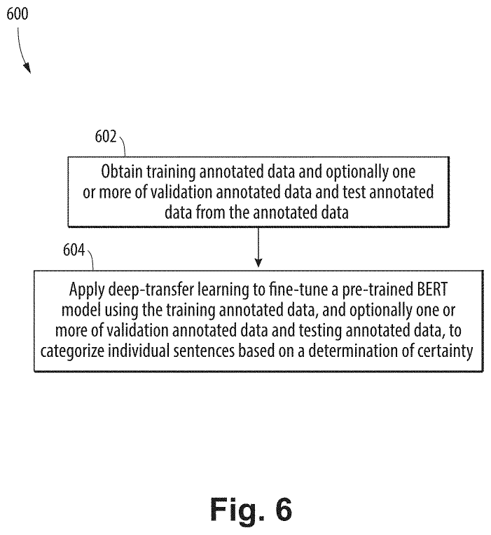

FIG. 6 shows a flowchart 600 of operations performed to train a language model, such as PTFT 106 shown in FIG. 1. At operation 602, training annotated data is obtained and optionally one or more of validation annotated data and testing annotated data from the annotated data. The training annotated data, validation annotated data, and testing annotated data can be, for example, training AD 222, validation AD 224, and testing AD 226 shown in FIG. 2. At operation 604, deep-transfer learning is applied to fine-tune a pre-trained BERT model using the training annotated data, as well as validation annotated data and test annotated data, in order to categorize individual sentences based on a determination of certainty.

Materials and methods used for applying and evaluating the disclosed method are described with respect to a study of radiology diagnostic reports, particularly for CT and MRI studies. While this example is directed to radiology diagnostics, and particularly to head MRI studies, the disclosure is not limited to analysis of radiology diagnostic reports, but can be applied to a variety of diagnostic reports (e.g., ultrasound diagnostic report, electrocardiogram diagnostic report, etc.). Furthermore, the generation of annotation data and fine-tuning of the PTFT model 106 can be performed for a particular context. Examples of different contexts in the field of radiology include various radiology imaging technologies (CT, positron emission technology (PET), ultrasound, etc.). Furthermore, the generation of annotation data and fine-tuning of the PTFT model 106 can be specific to a particular part of the anatomy or a particular technique used.

A PTFT model 106 that has already been pre-trained and fine-tuned for a first specific context can be re-trained by using a set of annotated data for second specific context to fine-tune the pre-trained BERT model in its original state before being fine-tuned for the first specific context. This re-training can be performed multiple times.

In one or more embodiments, re-training can be used to replace a previous training so that the PTFT model 106 is fine-tuned for only that specific context associated with the last re-training. This could be provided by reverting the PTFT model 106 back to the state it was in before being fine-tuned.

In one or more embodiments, the retraining can be used to add an additional specific context for which the PTFT model 106 can be used, in addition to the specific context associated with previous trainings. This could be provided by providing the PTFT model 106 with multiple pre-trained BERT models, wherein each pre-trained BERT model is fine-tuned for a different specific context. When submitting a diagnostic report, a user can select a specific context to use from several available specific contexts. For example, the user's user device can provide a graphical user interface (GUI) that provides a menu of specific contexts (e.g., head CT, head MRI, or pulmonary embolism CT diagnostic report) that can be used for assessing certainty of diagnostic reports.

### Example Method and Materials

An experiment was performed in which 594 randomly selected head MRI reports were presented to three board certified radiologists for assigning certainty levels to each sentence from impression sections of the reports. The 594 head MRI reports provided limited data. By employing a deep transfer learning approach to a deep neural networks-based knowledge representation that was pre-trained using a very large universal textual dataset, the specialized context of the 594 head MRI reports was transferred through fine-tuning the pre-trained neural networks. The objective was to unlock contextualized semantics of radiology reporting language at the sentence level, providing an effective solution to automatically qualify certainty expressed in the head MRI reports by assigning the certainty associated with each sentence into a different category of several available categories. This method provides an automatic measurement tool for measuring certainty quality of radiology reporting, both retrospectively and prospectively. This tool can include an interactive capability that has the potential to help improve communication of certainty associated with diagnostic reporting.

Example Annotation Process;

Three board-certified radiologists were asked to read sentences from the impression sections provided and to assign each sentence a certainty category from one of four possible certainty categories shown in Table 1:

TABLE 1
Categories for Annotation of Diagnostic Certainty of Diagnosis in the
Impression Section of a Radiology Report

| Certainty | | |
|---|---|---|
| Categories | Interpretation | Examples |
| Non-Definitive | Describing differential diagnoses without | Less likely differential considerations |
| | indicating any confidence or only findings | include demyelinating/inflammatory |
| | without any diagnosis. | processes. |
| Definitive-Strong | Describing discreet diagnostic findings | Stable right sphenoid intraosseous |
| | without hedging words. | lipoma. |
| Definitive-Mild | Describing discreet diagnostic findings | Findings suggestive of Arnold Chiari |
| | with hedging words. | 1 malformation. |
| Other | Describing recommendations, imaging | Another follow-up is recommended. |
| | techniques, prior studies. | |

“Diagnostic findings” are defined as a diagnostic opinion regarding a specific disease or other condition. The certainty category is not only dependent on hedging terms used, but is also based on a holistic context expressed in the sentence. Hedging terms can be perceived differently by various physicians, radiologists and patients, hence hedging terms were considered as only one factor of many that contributes to uncertainty.

Initially, the three annotators each reviewed 30 head MRI reports using a set of annotation rules and then applied an iterative process of adjusting the annotation rules and amending the annotations based on the adjusted annotation rules until a consensus was reached among the three annotators. Each annotator then independently annotated an additional 24 MRI head reports according to the last version of the annotation rules. An inter-rater agreement was calculated to be (0.74) as measured by Cohen's Kappa statistics, which showed substantial strength of agreement across annotators. Finally, each annotator annotated 180 MRI head reports, resulting in a total of 594 reports.

The annotated data was then analyzed for certainty qualification, as follows. Word tokenization and sentence boundary identification was performed on all the sentences from the impression section of the radiology reports. Sentences were removed that contained less than four words, as these short sentences are typically noise caused by sentence splitting errors, resulting in 2,352 sentences in total for further analysis. The annotated data was then split into certainty data into training annotation data (80%), validation annotation data (10%) and testing annotation data (10%). The training and validation annotation data were used for fine-tuning, and the testing data was used as “held-out” (unseen) data to evaluate performance. The data statistics for each dataset are shown in Table 2:

TABLE 2
Frequency of each Classification across Three Datasets: N(%).

| | Train_Dataset | Valid_Dataset | Test_Dataset |
|---|---|---|---|
| Non-Definitive | 585(30.97%) | 73(30.93%) | 73(30.8%) |
| Definitive-Mild | 329(17.42%) | 41(17.37%) | 42(17.7%) |
| Definitive-Strong | 503(26.63%) | 63(26.69%) | 63(26.58%) |
| Other | 472(24.97%) | 59(25%) | 59(24.89%) |
| Total | 1,889(100%) | 236(100%) | 237(100%) |

Deep Transfer Learning

The certainty assessment was handled as a multi-class sentence classification problem, and exploited NLP techniques to capture fine-grained semantics for classifying each sentence into one of the four categories defined in Table 1. Recent progress in NLP has been driven by using deep learning approaches. Different deep learning architectures have been applied for text classification, which typically can be grouped into two model families, for example convolutional neural networks (CNNs), which are good at extracting local and position-invariant pattern features, and recurrent neural networks (RNNs), which have been shown to perform better in modeling long dependencies among texts. Such deep learning approaches require large amounts of labeled (annotated) data in order to reliably estimate numerous model parameters; however, compared with general domains, annotated data are more difficult and expensive to obtain for clinical domains, as they require subject matter expertise for high quality annotation.

In this study, a NLP transferring learning model, BERT, was used and pre-trained. Pre-processing included extraction of data from an annotation tool and sentence segmentation using natural language toolkit (NLTK) such that each sentence was paired with a certainty label described in Table 2. A tokenizer (in this case WordPiece™ was used for tokenization for breaking down each word to its prefix, root, and suffix subwords.

The pre-training step enables BERT to learn deep and intimate understandings of how a particular language works from large volumes of unlabeled plain text data (meaning in an unsupervised manner). The pre-trained BERT was then imported in order to fine-tune BERT's parameters to build a task-specific model. It is noted that BERT reads an entire sequence of words at once by reading bidirectionally. This characteristic allows BERT to learn the context of a word based on its surroundings within a sentence. Each sequence of words (e.g., a sentence) is encoded into a numeric vector that represents meaning of the sequence of word's for the subsequent classification task.

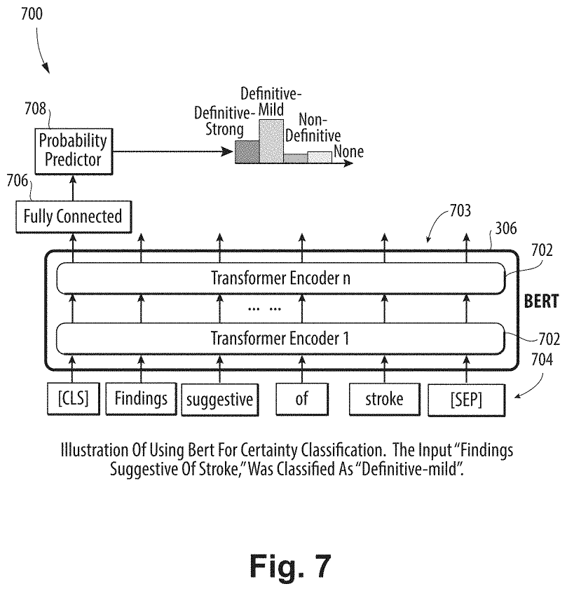

With reference to FIG. 7, an example flow diagram 700 for processing an input sentence 704 “Findings suggestive of stroke” that is composed of a sequence of four token (words) during fine-tuning of a pre-trained BERT model 306. During pre-processing, for compatibility with BERT, a special classification token [CLS] is added at the beginning of the input sentence 704, and another special token [SEP] is added at the end of the input sentence 704 to separate multiple sentences, if any. The input sentence 794 is input to encoders 702 of a pre-trained BERT model 306. Parameters, including a fully connected layer 706, of the pre-trained BERT model 306 are fine-tuned through supervised learning using the annotated data for certainty classification. Through a multi-layer deep neural network architecture having n transformer blocks 702 of an encoder 703 of the pre-trained BERT model 306, each input token is transformed to a final hidden state (e.g., vector representation). For this sentence classification task, only the final hidden state of the first token, [CLS], which represents classification of the aggregated sentence, is provided to a fully-connected layer 706 to obtain a probability distribution over four certainty categories through a probability distribution function used by a probability processor 708.

Experimentation was performed using three variants of pre-trained BERT models: (1) BERT-base which consists of an encoder with 12 (n=12 in FIG. 7) layer transformer blocks, and was pre-trained using BookCorpus and WIKIPEDIA™; (2) BioBERT which was initialized using BERT-base and was pre-trained using BookCorpus, WIKIPEDIA, PUBMED™ abstracts and PMC (PUBMED Central) full text articles; (3) ClinicalBERT which was initialized using BioBERT and pre-trained using about two million of clinical notes in MIMIC-III database.

Results

Classification performance was reported against reference standard categories assigned by radiologists using standard metrics: sensitivity, specificity and area under the receiver operating characteristic curve (ROC-AUC). The aggregated results are presented with the macro-average across the four categories defined in Table 1. The macro-average is a calculation of each metric (e.g., sensitivity, specificity or ROC-AUC) independently for each category, for which an average is then determined. For three pre-trained language models, a grid search was used to optimize batch size (range used: [24,32,64]) and learning rate (range used: [0.000005, 0.00001, 0.00003, 0.00005]) during the fine-tuning process, for a fair comparison. A number of epochs for fine-tuning training was selected based on a peak AUC score for the validation annotated data.

Comparative Performance Among Three BERT Models (Validation Data)

Performance of certainty classification by the three described pre-trained BERT models was compared, with results shown in Table 3. As shown, the bioBERT model obtained the best macro ROC-AUC of 0.931, and BERT-base yielded the best macro sensitivity of 79.46% and specificity of 93.65%, while ClinicalBERT achieved the relatively lower macro sensitivity of 78.52% compared with the other two models:

TABLE 3
Performance Comparison among Three BERT Variants

| | # of | Batch | Learning | Macro- | Macro- | Macro-ROC- |
|---|---|---|---|---|---|---|
| Model | Epoch | Size | Rate | Sensitivity | Specificity | AUC |
| BERT-base | 4 | 24 | 0.00003 | 79.46% | 93.65% | 0.928 |
| | | | | [68.02, 87.82] | [89.26, 96.46] | [0.883, 0.973] |
| BioBERT | 6 | 32 | 0.00003 | 79.08% | 93.13% | 0.931 |
| | | | | [67.13, 87.78] | [88.58, 96.13] | [0.886, 0.975] |
| ClinicalBERT | 5 | 32 | 0.00005 | 78.52% | 93.19% | 0.925 |
| | | | | [66.91, 87.07] | [88.57, 96.25] | [0.878, 0.971] |

Note:
95% CIs are shown in brackets
Macro = Average on the macro level across different categories
ROC = Receiver Operating Characteristics
AUC = Area under the ROC curve

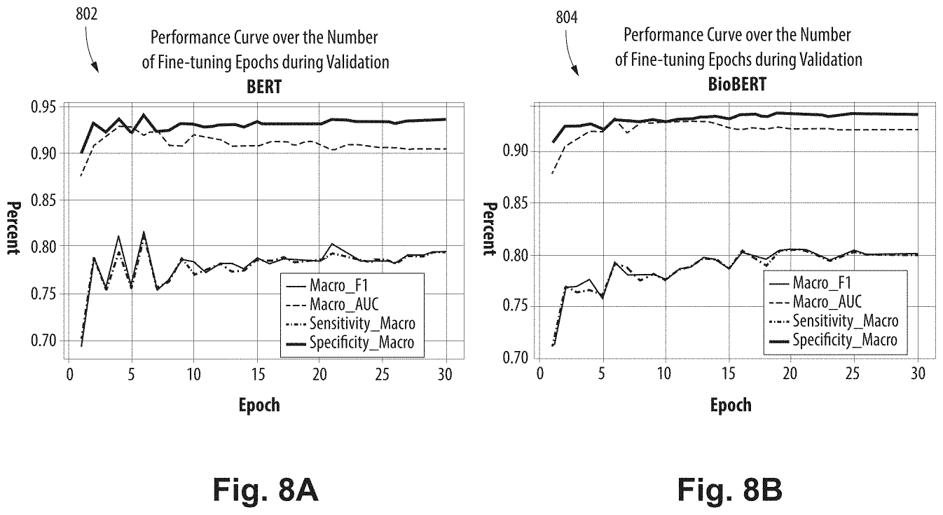

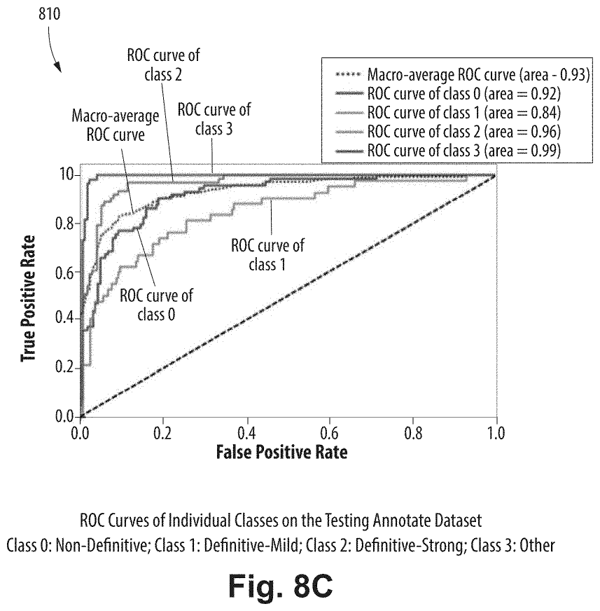

Performance Curve in the Fine-tuning Process (Validation Annotated Data) FIG. 8A shows first performance curves 802 of BERT-base and FIG. 8B shows second performance curves 804 of bioBERT over a number of epochs during the fine-tuning process. An F1 score is also shown, which is the harmonic mean of positive predictive value and sensitivity. Similar trends were observed on both models. With fine-tuning, the performance metrics increase initially and then plateaued after approximately five epoch trainings. This was true for all performance metrics tracked.

Performance on the Testing Data

Based on the evaluation results (AUC scores) on the validation annotation data, the best performing system (BioBERT) was chosen and applied to the test annotation data as shown in Table 4:

TABLE 4
System Performance on the Testing Annotated Dataset

| Category | Sensitivity | Specificity | ROC-AUC |
|---|---|---|---|
| Non-Definitive | 76.71% (56/73) | 90.24% (148/164) | 0.919 |
| | [65.35, 85.81] | [84.64, 94.32] | [0.874, 0.964] |
| Definitive-Mild | 59.52% (25/42) | 88.72% (173/195) | 0.843 |
| | [43.28, 74.37] | [83.42, 92.79] | [0.766, 0.92] |
| Definitive-Strong | 74.6% (47/63) | 95.4% (166/174) | 0.964 |
| | [62.06, 84.73] | [91.14, 97.99] | [0.931, 0.997] |
| Other | 98.31% (58/59) | 97.19% (173/178) | 0.994 |
| | [90.91, 99.96] | [93.57, 99.08] | [0.979, 1] |
| Macro Avg. | 77.29% | 92.89% | 0.93 |
| | [65.4, 86.22] | [88.19, 96.05] | [0.888, 0.972] |

Note:
numerators and denominators for sensitivity and specificity are included in parentheses
95% CIs are shown in brackets
ROC = Receiver Operating Characteristics
AUC = Area under the ROC curve
Macro Avg = Average on the macro level across different categories

The system represented in Table 4 performs best on the “Other” category, which has a highest sensitivity of 98.31%, specificity of 97.19%, and ROC-AUC of 0.994. Among the other three categories, the system represented in Table 4 obtained the highest sensitivity for Non-Definitive (76.71%), the highest specificity for Definitive-Strong (95.4%), and the highest ROC-AUC for Definitive-Strong (0.964). Overall, this system obtained the macro average Sensitivity of 77.29%, Specificity of 92.89% and ROC-AUC of 0.93 on the held-out unseen testing annotated data. Although the “Non-Definitive” class has a lower ROC-AUC score than “Definitive-Strong,” the Sensitivity of “Non-Definitive” is better than “Definitive-Strong” (76.71% vs. 74.6%) as shown in Table 4. As shown in FIG. 8C, ROC-AUC curves 810 of “Definitive-Strong” (class 2) and “Other” (class 3) are closer to an ideal spot (wherein “ideal” is based on proximity to the upper left corner).

Error Analysis

Error analysis conducted on the validation annotated data and a confusion matrix is shown in Table 5:

TABLE 5
Confusion Matrix Among Different Categories

| | | Prediction | | | |
|---|---|---|---|---|---|
| | | Non-Definitive | Definitive-Mild | Definitive-Strong | Other |
| Truth | Non-Definitive | 56 | 11 | 3 | 3 |
| | Definitive-Mild | 11 | 28 | 2 | 0 |
| | Definitive-Strong | 11 | 5 | 46 | 1 |
| | Other | 1 | 0 | 0 | 58 |

The rows in Table 5 represent truth labels assigned by domain experts, and the columns represent system predictions. Table 5 shows that only one (1.7%) sentence in the “Other” category was wrongly classified as “Non-Definitive,” which explains the high performance in this category shown in Table 4. Based on the definition of “Other” category, it covers a narrow scope of semantics which is easy for the system employed to pick up representative patterns (e.g., “follow up” is a reliable indicator for recommendations). Top three error patterns are: (1) “Non-Definitive×Definitive-Mild” (11 out of 73, 15%); (2) “Definitive-Mild×Non-Definitive” (11 out of 41, 26.8%); (3) “Definitive-Strong×Non-Definitive” (11 out of 63, 17.5%). This suggests that the “Non-Definitive” category is more challenging to be distinguished from the other two definitive categories.

The above study indicates that certainty of each sentence in the impression section of radiology reports can be categorized into different certainty categories. The disclosed automated categorization scheme was shown to have strong operating characteristics when compared with a “ground truth” based on radiologist consensus.

The disclosed diagnostic report processing system 100 can provide a diagnostician with a real-time automatic measurement of a level of diagnostic certainty in a diagnostic report authored by the diagnostician and/or a prompt to correct certainty confusion prior to submitting the diagnostic report for recordation or further usage. In this way, the diagnostician can have objective information about the level of certainty that is being conveyed. Sentences of diagnosis explanations and/or complete diagnosis explanations can be assigned to a discrete certainty category and/or assigned a probability that the assigned certainty level is correct. The assigned certainty category can be used to determine treatment of the diagnostic evaluation, to provide a quality metric to evaluate a diagnostician's performance, and/or to determine treatment of future diagnostic evaluations by the diagnostician (such as whether future diagnostic evaluations by the diagnostician a marked as questionable or require a supervisory review before being stored).

An external validation included a random sampling of a new set of 40 MRI head reports from four new neuroradiologists (ten reports each) who were not consulted for the original data collection. The original three radiologists used for the original data collection were asked to assign one of the four certainty categories to all a new data set including 132 sentences from impression sections of these 40 reports. It was found that inter-annotator agreement remained very high, with a mean pairwise Kappa score of 0.761. Annotations were chosen from the annotator who agreed most with the other two annotators as a ground truth. Performance of diagnostic report manager 102 was evaluated for the new dataset, achieving macro sensitivity of 84.01%, macro specificity of 93.59%, and macro AUROC of 0.945, which demonstrates great generalizability of the diagnostic report manager 102 and ground truth consensus.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the illustrated embodiments, exemplary methods and materials are now described. All publications mentioned herein are incorporated herein by reference to disclose and describe the methods and/or materials in connection with which the publications are cited.

It must be noted that as used herein and in the appended claims, the singular forms “a,” “an,” and “the” include plural referents unless the context clearly dictates otherwise. Thus, for example, reference to “a stimulus” includes a plurality of such stimuli and reference to “the signal” includes reference to one or more signals and equivalents thereof known to those skilled in the art, and so forth.

It is to be appreciated the embodiments of the disclosure include software algorithms, programs, or code that can reside on a computer useable medium having control logic for enabling execution on a machine having a computer processor. The machine typically includes memory storage configured to provide output from execution of the computer algorithm or program.

As used herein, the term “software” is meant to be synonymous with any code or program that can be in a processor of a host computer, regardless of whether the implementation is in hardware, firmware or as a software computer product available on a disc, a memory storage device, or for download from a remote machine. The embodiments described herein include such software to implement the logic, equations, relationships and algorithms described above. One skilled in the art will appreciate further features and advantages of the illustrated embodiments based on the above-described embodiments. Accordingly, the illustrated embodiments are not to be limited by what has been particularly shown and described, except as indicated by the appended claims.

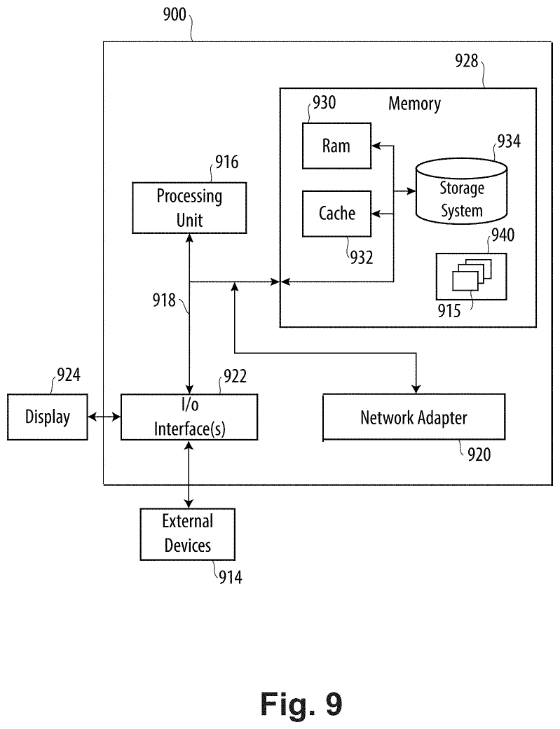

Embodiments of the components of diagnostic report processing system 100, such as diagnostic report manager 102, annotation processor 140, and assessor device 206 as well as the models, including BERT model 302, pre-trained BERT model 306, and PTFT model 106, may be implemented or executed by one or more computer systems, such as example computer system 900 illustrated in FIG. 9. The components of diagnostic report processing system 100 can share resources, including hardware and software resources.

Each computer system 900 can implement one or more components or models of diagnostic report processing system 100 or multiple instances thereof. In various embodiments, computer system 900 may include a server, a mainframe computer system, a workstation, a network computer, a desktop computer, a laptop, or the like, and/or include one or more of a field-programmable gate array (FPGA), application specific integrated circuit (ASIC), microcontroller, microprocessor, or the like.

Computer system 900 is only one example of a suitable system and is not intended to suggest any limitation as to the scope of use or functionality of embodiments of the disclosure described herein. Regardless, computer system 900 is capable of being implemented and/or performing any of the functionality set forth hereinabove.

Computer system 900 may be described in the general context of computer system-executable instructions, such as program modules, being executed by a computer system. Generally, program modules may include routines, programs, objects, components, logic, data structures, and so on that perform particular tasks or implement particular abstract data types. Computer system 900 may be practiced in distributed data processing environments where tasks are performed by remote processing devices that are linked through a communications network. In a distributed data processing environment, program modules may be located in both local and remote computer system storage media including memory storage devices.

Computer system 900 is shown in FIG. 9 in the form of a general-purpose computing device. The components of computer system 900 may include, but are not limited to, one or more processors or processing units 916, a system memory 928, and a bus 918 that couples various system components including system memory 928 to processor 916. Bus 918 represents one or more of any of several types of bus structures, including a memory bus or memory controller, a peripheral bus, an accelerated graphics port, and a processor or local bus using any of a variety of bus architectures. By way of example, and not limitation, such architectures include Industry Standard Architecture (ISA) bus, Micro Channel Architecture (MCA) bus, Enhanced ISA (EISA) bus, Video Electronics Standards Association (VESA) local bus, and Peripheral Component Interconnect (PCI) bus.

Computer system 900 typically includes a variety of computer system readable media. Such media may be any available media that is accessible by the components or models of diagnostic report processing system 100, and it includes both volatile and non-volatile media, removable and non-removable media.

System memory 928 can include computer system readable media in the form of volatile memory, such as random access memory (RAM) 930 and/or cache memory 932. Computer system 900 may further include other removable/non-removable, volatile/non-volatile computer system storage media. By way of example only, storage system 934 can be provided for reading from and writing to a non-removable, non-volatile magnetic media (not shown and typically called a “hard drive”). Although not shown, a magnetic disk drive for reading from and writing to a removable, non-volatile magnetic disk, and an optical disk drive for reading from or writing to a removable, non-volatile optical disk such as a CD-ROM, DVD-ROM or other optical media can be provided. In such instances, each can be connected to bus 918 by one or more data media interfaces. As will be further depicted and described below, memory 928 may include at least one program product having a set (e.g., at least one) of program modules that are configured to carry out the functions of embodiments of the disclosure.

Program/utility 940, having a set (at least one) of program modules 915, such as for performing the operations of flowcharts 400, 500, and 600 shown in FIGS. 4-6, respectively, may be stored in memory 928 by way of example, and not limitation, as well as an operating system, one or more application programs, other program modules, and program data. Each of the operating system, one or more application programs, other program modules, and program data or some combination thereof, may include an implementation of a networking environment. Program modules 915 generally carry out the functions and/or methodologies of embodiments of the disclosure as described herein.

Computer system 900 may also communicate with one or more external devices 914 such as a keyboard, a pointing device, a display 924, etc.; one or more devices that enable a user to interact with computer system 900; and/or any devices (e.g., network card, modem, etc.) that enable the components or models of diagnostic report processing system 100 to communicate with one or more other computing devices. Such communication can occur via Input/Output (I/O) interfaces 922. Still yet, computer system 900 can communicate with one or more networks such as a local area network (LAN), a general wide area network (WAN), and/or a public network (e.g., the Internet) via network adapter 920. As depicted, network adapter 920 communicates with the other components or models of diagnostic report processing system 100 via bus 918. It should be understood that although not shown, other hardware and/or software components could be used in conjunction with computer system 900. Examples, include, but are not limited to: microcode, device drivers, redundant processing units, external disk drive arrays, RAID systems, tape drives, and data archival storage systems, etc.

The flow diagram and block diagrams in the Figures illustrate the architecture, functionality, and operation of possible implementations of systems, methods and computer program products according to various embodiments of the present disclosure. In this regard, each block in the flow diagram or block diagrams may represent a module, segment, or portion of code, which comprises one or more executable instructions for implementing the specified logical function(s). It should also be noted that, in some alternative implementations, the functions noted in the block may occur out of the order noted in the figures. For example, two blocks shown in succession may, in fact, be executed substantially concurrently, or the blocks may sometimes be executed in the reverse order, depending upon the functionality involved. It will also be noted that each block of the block diagrams and/or flow diagram illustration, and combinations of blocks in the block diagrams and/or flow diagram illustration, can be implemented by special purpose hardware-based systems that perform the specified functions or acts, or combinations of special purpose hardware and computer instructions.

The descriptions of the various embodiments of the present disclosure have been presented for purposes of illustration, but are not intended to be exhaustive or limited to the embodiments disclosed. Many modifications and variations will be apparent to those of ordinary skill in the art without departing from the scope and spirit of the described embodiments. The terminology used herein was chosen to best explain the principles of the embodiments, the practical application or technical improvement over technologies found in the marketplace, or to enable others of ordinary skill in the art to understand the embodiments disclosed herein.

A potential advantage that can be gained via the various embodiments of interactions between the components and models of diagnostic report processing system 100 disclosed include leveraging deep transfer learning to understand contextualized semantics and use this information for certainty classification using limited annotated data. A further potential advantage is automating assessment of uncertainty in the context of diagnostic reports, e.g., radiology, reports to help facilitate increased precision communication of diagnostic findings. The automatically generated assessment of uncertainty can be used to interactively prompt a user to provide a diagnostic report that prompts a diagnostician to edit a diagnostic report until it satisfies certainty criteria, to control treatment of the diagnostic report, and/or to evaluate the diagnostician that authored the diagnostic report.

While the apparatus and methods of the subject disclosure have been shown and described with reference to preferred embodiments, those skilled in the art will readily appreciate that changes and/or modifications may be made thereto without departing from the spirit and scope of the subject disclosure.

What is claimed is:

1. A method for assessing diagnostic certainty in diagnostic reporting natural language, the method comprising:
receiving an impression portion of a diagnostic report submitted for certainty evaluation, the impression portion having one or more sentences of natural language;
accessing a trained language model that was:
trained in a pre-training stage in an unsupervised manner using artificial intelligence-based deep neural network learning by deep bidirectional reading of a large amount of word sequences of first training sentences of natural language and outputting a bidirectional language model, wherein the language model is configured to predict one or more words from their respective bidirectional context and/or to transform natural language input into a latent semantic space to represent the respective one or more words based on bi-directionally surrounding words; and
further trained in a fine-tuning stage for evaluating certainty of a small amount of training impression portions of diagnostic reports specific to a task, the training impression portions including a plurality of one or more second training sentences of natural language, the evaluating certainty of the training impression portions including generating certainty data per second training sentence indicative of a result of applying annotation rules specific to the task based on context provided by the second training sentence as a whole;
applying the one or more sentences to the trained language model for evaluation of the one or more sentences as a whole;
receiving an assessment of certainty for the respective one or more sentences based on the evaluation;
communicating the assessment of certainty to a user before accepting the impression portion; and
accepting submission of the impression portion only after the impression portion satisfies certainty criteria, or if the certainty criteria is not required, obtaining validation from the user.

2. The method of claim 1, further comprising generating the assessment of certainty, wherein the assessment of certainty includes assignment to a certainty category of a plurality of certainty category, each certainty category indicating a different level or type of certainty.

3. The method of claim 2, wherein the generating the assessment of certainty further includes determining a probability that the assignment to the certainty category is correct.

4. The method of claim 1, further comprising, for an assessment of certainty that fails to satisfy a certainty criteria, providing an opportunity to update the impression portion and resubmitting the impression portion for application of the one or more sentences of the updated impression portion to the trained language model.

5. The method of claim 1, further comprising training the trained language model in the fine-tuning stage using a training set that is a subset of the certainty data.

6. The method of claim 5, wherein training the trained language model in the fine-tuning stage includes at least one of validating using a validation set that is a subset of the certainty data and testing using a testing set that is a subset of the certainty data.

7. The method of claim 5, wherein training the trained language model in the fine-tuning stage includes iteratively adjusting at least one of the annotation rules and the certainty data by a plurality of reviewers until evaluation of same second training sentences by the plurality of reviewers results in the certainty data generated by different reviewers of the plurality of reviewers satisfying a criterion of consensus.

8. The method claim 5, further comprising retraining the trained language model in the fine-tuning stage, based on a state of at least a portion of the trained language model after the pre-training stage and before the fine-tuning stage, using a second small amount of second training impression portions of diagnostic reports specific to a second task, the second training impression portions including a plurality of one or more third training sentences of natural language, the evaluating certainty of the second training impression portions including generating second certainty data per third training sentence indicative of a result of applying annotation rules specific to the second task based on context provided by the third training sentence as a whole.

9. A method for assessing diagnostic certainty in radiology reporting natural language, the method comprising:
receiving an impression portion of a radiology report submitted for certainty evaluation, the impression portion having one or more sentences of natural language;
accessing a trained language model that was:
trained in a pre-training stage in an unsupervised manner using artificial intelligence-based deep neural network learning by deep bidirectional reading of a large amount of word sequences of first training sentences of natural language and outputting a measurement of certainty per first training sentence; and
further trained in a fine-tuning stage for evaluating certainty of a small amount of training impression portions of diagnostic reports specific to a task, the training impression portions including a plurality of one or more second training sentences of natural language, the evaluating certainty of the training impression portions including generating certainty data per second training sentence indicative of a result of applying annotation rules specific to the task based on context provided by the second training sentence as a whole;
applying the one or more sentences to the trained language model for evaluation of the one or more sentences as a whole;
receiving an assessment of certainty for the respective one or more sentences based on the evaluation;
communicating the assessment of certainty to a user before accepting the impression portion; and
accepting submission of the impression portion only after the impression portion satisfies certainty criteria, or if the certainty criteria is not required obtaining validation from the user.

10. A computer system for managing threats to a network, comprising:
a memory configured to store instructions;
processor disposed in communication with said memory, wherein the processor upon execution of the instructions is configured to:
receive an impression portion of a diagnostic report submitted for certainty evaluation, the impression portion having one or more sentences of natural language;
access a trained language model that was:
trained in a pre-training stage in an unsupervised manner using artificial intelligence-based deep neural network learning by deep bidirectional reading of a large amount of word sequences of first training sentences of natural language and outputting a measurement of certainty per first training sentence; and
further trained in a fine-tuning stage for evaluating certainty of a small amount of training impression portions of diagnostic reports specific to a task, the training impression portions including a plurality of one or more second training sentences of natural language, the evaluating certainty of the training impression portions including generating certainty data per second training sentence indicative of a result of applying annotation rules specific to the task based on context provided by the second training sentence as a whole;
apply the one or more sentences to the trained language model for evaluation of the one or more sentences as a whole;
receive an assessment of certainty for the respective one or more sentences based on the evaluation;
communicate the assessment of certainty to a user before accepting the impression portion; and
accept submission of the impression portion only after the impression portion satisfies certainty criteria, or if the certainty criteria is not required obtaining validation from the user.

11. The computer system of claim 10, wherein the processor upon execution of the instructions is further configured to generate the assessment of certainty, wherein the assessment of certainty includes assignment to a certainty category of a plurality of certainty category, each certainty category indicating a different level or type of certainty.

12. The computer system of claim 11, wherein the generating the assessment of certainty further includes determining a probability that the assignment to the certainty category is correct.

13. The computer system of claim 10, wherein for an assessment of certainty that fails to satisfy a certainty criteria, the processor upon execution of the instructions is further configured to provide an opportunity to update the impression portion and resubmit the impression portion for application of the one or more sentences of the updated impression portion to the trained language model.

14. The computer system of claim 10, wherein the processor upon execution of the instructions is further configured to train the trained language model in the fine-tuning stage using a training set that is a subset of the certainty data.

15. The computer system of claim 14, wherein training the trained language model in the fine-tuning stage includes at least one of validating using a validation set that is a subset of the certainty data and testing using a testing set that is a subset of the certainty data.

16. The computer system of claim 14, wherein training the trained language model in the fine-tuning stage includes iteratively adjusting at least one of the annotation rules and the certainty data by a plurality of reviewers until evaluation of same second training sentences by the plurality of reviewers results in the certainty data generated by different reviewers of the plurality of reviewers satisfying a criterion of consensus.

17. The computer system of claim 14, wherein the processor upon execution of the instructions is further configured to retrain the trained language model in the fine-tuning stage, based on a state of at least a portion of the trained language model after the pre-training stage and before the fine-tuning stage, using a second small amount of second training impression portions of diagnostic reports specific to a second task, the second training impression portions including a plurality of one or more third training sentences of natural language, the evaluating certainty of the second training impression portions including generating second certainty data per third training sentence indicative of a result of applying annotation rules specific to the second task based on context provided by the third training sentence as a whole.

18. A non-transitory computer readable storage medium and one or more computer programs embedded therein, the computer programs comprising instructions, which when executed by a computer system, cause the computer system to:
receive an impression portion of a diagnostic report submitted for certainty evaluation, the impression portion having one or more sentences of natural language;
access a trained language model that was:
trained in a pre-training stage in an unsupervised manner using artificial intelligence-based deep neural network learning by deep bidirectional reading of a large amount of word sequences of first training sentences of natural language and outputting a measurement of certainty per first training sentence; and
further trained in a fine-tuning stage for evaluating certainty of a small amount of training impression portions of diagnostic reports specific to a task, the training impression portions including a plurality of one or more second training sentences of natural language, the evaluating certainty of the training impression portions including generating certainty data per second training sentence indicative of a result of applying annotation rules specific to the task based on context provided by the second training sentence as a whole;
apply the one or more sentences to the trained language model for evaluation of the one or more sentences as a whole;
receive an assessment of certainty for the respective one or more sentences;
communicate the assessment of certainty to a user before accepting the impression portion; and
accept submission of the impression portion only after the impression portion satisfies certainty criteria, or if the certainty criteria is not required obtaining validation from the user.

19. The non-transitory computer readable storage medium of claim 18, wherein the computer programs instructions that when executed by a computer system further cause the computer system to generate the assessment of certainty, wherein the assessment of certainty includes assignment to a certainty category of a plurality of certainty category, each certainty category indicating a different level or type of certainty.

20. The non-transitory computer readable storage medium 14, wherein the computer programs instructions that when executed by a computer system further cause the computer system to train the trained language model in the fine-tuning stage using a training set that is a subset of the certainty data, wherein training the trained language model in the fine-tuning stage includes iteratively adjusting at least one of the annotation rules and the certainty data by a plurality of reviewers until evaluation of same second training sentences by the plurality of reviewers results in the certainty data generated by different reviewers of the plurality of reviewers satisfying a criterion of consensus.
